(19) United States
(12) Patent Application Publication
(10) Pub. No.: US 2013/0295083 A1
(43) Pub. Date: Nov. 7, 2013

(54) 2a-Methyl-19-nor-(20S)-1a,25-dihydroxyvitamin D3 (2AMD) or 2 methylene-19-nor-(20S)-1a,25-dihydroxyvitamin D3 (2MD) Support Survival and Function of Transplanted Islet Cells In Type 1 Diabetes

(71) Applicant: Wisconsin Alumni Research Foundation, Madison, WI (US)

(72) Inventors: Hector F. DeLuca, Deerfield, WI (US); Carol M. Kiekhaefer, Middleton, WI (US)

(73) Assignee: Wisconsin Alumni Research Foundation, Madison, WI (US)

(21) Appl. No.: 13/772,010

(22) Filed: Feb. 20, 2013

Related U.S. Application Data
(60) Provisional application No. 61/641,538, filed on May 2, 2012.

(51) Int. Cl. A61K 31/593; A61K 45/06

(52) U.S. Cl. CPC A61K 31/593; A61K 45/06
USPC 424/133.1; 514/167; 424/278.1; 552/653

(57) ABSTRACT

A method of treating islet cell transplant patients is described herein. The method comprises the steps of a) identifying an islet cell transplant patient, b) treating the patient with 2AMD or 2AMD analog and c) observing a prolonged period of normal glycemia is disclosed. A composition for treatment of islet cell transplant patient is disclosed as well, wherein the composition comprises 50-400 ng/day of 2AMD or 2AMD analog.

### CROSS-REFERENCE TO RELATED APPLICATIONS

This application claims the benefit of U.S. Provisional Patent Application No. 61/641,538, filed May 2, 2012, which is incorporated herein by reference in its entirety for all purposes.

### STATEMENT REGARDING FEDERALLY SPONSORED RESEARCH OR DEVELOPMENT

Not applicable.

### BACKGROUND

Type 1 diabetes (T1D) is a chronic autoimmune disorder resulting in the destruction of pancreatic beta islet cells. This destruction is followed by loss of insulin secretion and a lifelong dependence on insulin injections. Despite frequent glucose monitoring and intense insulin therapy, exogenous insulin cannot eliminate glycemic instability and its clinical consequences. Alternatively, successful islet cell transplantation provides stable control of blood glucose and can slow or prevent the progression of complications associated with diabetes, such as heart disease, kidney disease, and nerve or eye damage.

In islet transplantation, islets are taken from the pancreas of a deceased organ donor. The islets are purified, processed, and transferred into a diabetic recipient. Once implanted, the beta cells in these islets begin to make and release insulin. Restoration of normal insulin secretion in T1D patients reduces multi organ complications and improves quality of life.

The benefits of islet cell transplantation are currently limited by the fact that more than 90% of islet cell recipients revert to insulin dependence within 5 years after islet cell transplantation. The autoimmune response that destroyed the islet recipients' islets in the first place can recur and attack the transplanted islets. Although immunosuppressive drugs are required to keep the transplanted islets functioning, these drugs do not treat autoimmune destruction. There is a critical need to develop new treatment regimens that can control islet autoimmunity and recurrent diabetes in islet cell recipients.

### BRIEF SUMMARY OF THE INVENTION

The present invention is broadly summarized as relating to a method of treating islet cell transplant patients with a sufficient amount of 2AMD or 2AMD analog, as well as compositions for such treatments.

In a first aspect, the present invention provides a method of treating islet cell transplant patients. Such method comprises the steps of a) identifying an islet cell transplant patient, b) treating the patient with a sufficient amount of 2AMD or 2AMD analog such that a prolonged period of normoglycemia occurs compared to a patent who has not been treated; and c) observing a prolonged period of normoglycemia.

In one embodiment of the first aspect, the 2AMD or 2AMD analog is selected from the group of 2AMD and 2MD.

In another embodiment of the first aspect, the prolonged period is at least 1 year after transplant compared to an untreated control. Preferably, the prolonged period is at least 2 years. More preferably, the prolonged period is at least 3 years.

In another embodiment of the first aspect, the method comprises the additional step of treating the patient with immunosuppressants.

In another embodiment of the first aspect, the serum calcium level of the patient does not exceed 10.8 mg/dl during the treatment of step (b).

In a second aspect, the present invention provides a composition for treatment of islet cell transplant patient comprising 50-400 ng/day of 2AMD or 2AMD analog.

In a third aspect, the present invention provides a composition for treatment of islet cell transplant patient comprising 50-400 ng/day of 2AMD or 2AMD analog and at lease one immunosuppressant.

### BRIEF DESCRIPTION OF THE DRAWINGS

FIG. 1 is a graph of average weekly glucose versus weeks following islet cell transplantation.

FIG. 2 is a graph of serum calcium vs. duration following islet cell transplantation.

FIG. 3 is a graph of body weight versus weeks following islet cell transplantation.

FIG. 4 is a graph of average weekly glucose versus weeks following islet cell transplantation.

FIG. 5 is a graph of serum calcium vs. duration following islet cell transplantation.

FIG. 6 is a graph of body weight versus duration following islet cell transplantation.

### DESCRIPTION OF THE INVENTION

### In General

Our work involves the use of 2α-methyl-19-nor-(20S)-1α,25-dihydroxyvitamin D₃ (2AMD) or 2-methylene-19-nor-(20S)-1α,25-dihydroxyvitamin D₃ (2MD) as a selective therapy for achieving long-term engraftment and tolerance of transplanted islets for patients with autoimmune diabetes. Preclinical results in a rodent model demonstrate that 2AMD has immunotherapeutic effects against autoimmune destruction of transplanted islets.

We previously published the results of studies examining the immunoprotective effects of 2AMD (C M Kiekhaefer et. al., 2α-Methyl-19-nor-(20S)-1,25-dihydroxyvitamin D₃ protects the insulin-2 knockout non-obese diabetic mouse from developing type 1 diabetes without hypocalcaemia, Clinical and Experimental Immunology, 2011 December; 166 (3): 325-32). Using a rodent model of spontaneous T1D, we demonstrated that treatment with 2AMD preserved islet cell structure and function, arrested T cell invasion, and prevented the transition from insulitis to diabetes.

Our more recent studies, reported herein, have assessed the potential of 2AMD as a selective therapy for achieving long-term engraftment and tolerance in islet cell recipients. Preliminary results in a rodent model of spontaneous diabetes suggest that the analog has immunotherapeutic effects against autoimmune destruction of transplanted islets. Further investigations, also reported below, have demonstrated the benefits of combining 2AMD treatment with a purified diet to improve long-term islet engraftment under conditions using a significantly reduced marginal mass of islet cells. The relevance of these findings is useful to the reversal of hyperglycemia in human diabetic recipients where availability of islets from cadaver donors is limited.

Overall, these investigations demonstrate the efficacy of 2AMD and related compounds for inhibiting autoimmune destruction of transplanted islets and the potential for reversal of hyperglycemia with reduced islet mass.

A closely related analog of 2AMD is 2-methylene-19-nor-(20S)-1α,25-dihydroxyvitamin D₃ (2MD), which has almost identical activities to 2AMD. We expect 2MD to be equally effective as 2AMD in the protection of islet cell survival. Additionally, we envision that other 2AMD analogs will be effective. By “2AMD analogs”, we mean to include the 2AMD analogs listed below in Table 1:

TABLE 1

| | |
|---|---|
| 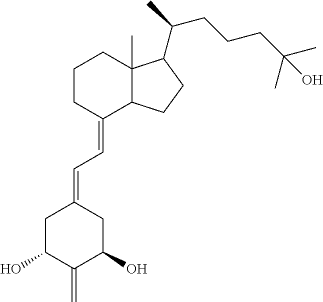 [SMILES: C=C1[C@H](O)CC(=C/C=C2\CCCC3(C)C2CCC3[C@@H](C)CCCC(C)(C)O)C[C@H]1O] | 2MD |
| 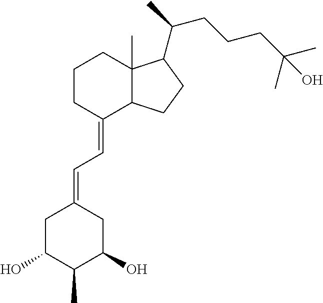 | 2AMD |
| 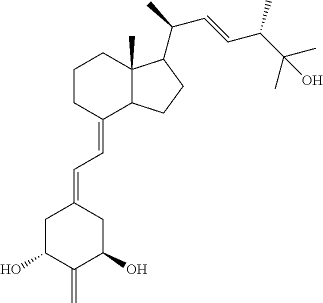 [SMILES: C=C1[C@H](O)CC(=C/C=C2\CCC[C@@]3(C)C2CCC3[C@@H](C)/C=C/[C@H](C)C(C)(C)O)C[C@H]1O] | (20S/24S)2- MD₂ |
| 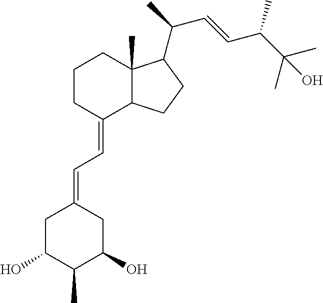 | (20S/24S)2- AMD₂ |
| 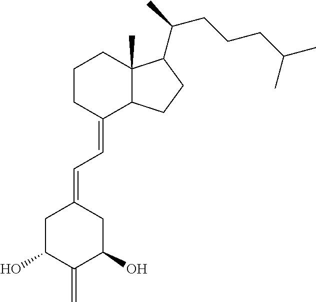 [SMILES: C=C1[C@H](O)CC(=C/C=C2\CCC[C@@]3(C)C2CCC3[C@@H](C)CCCC(C)C)C[C@H]1O] | 2M-1αOH PN-1 |
| 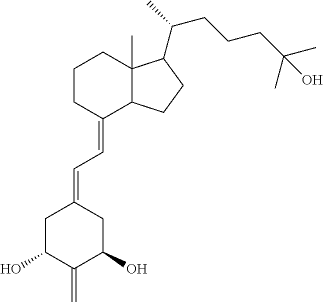 [SMILES: C=C1[C@H](O)CC(=C/C=C2\CCCC3(C)C2CCC3[C@H](C)CCCC(C)(C)O)C[C@H]1O] | 20R-2MD |
| 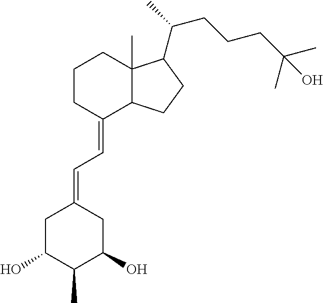 | 20R-2AMD |
| 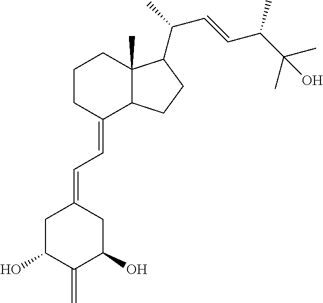 [SMILES: C=C1[C@H](O)CC(=C/C=C2\CCC[C@@]3(C)C2CCC3[C@H](C)/C=C/[C@H](C)C(C)(C)O)C[C@H]1O] | (20R/24S)2- MD2 |
| 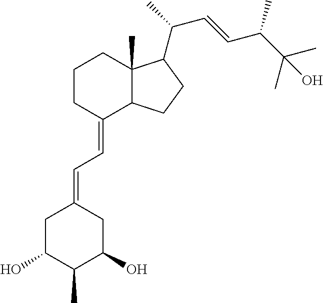 | (20R/24S)2- AMD2 |

### Method of Treating Islet Cell Transplant Patients

In one aspect of the present invention 2AMD or a 2AMD analog is used to improve the duration and efficiency of an islet cell transplant. Preferably, one would treat a patient with either 2AMD or a specific 2AMD analog and would not combine different compounds.

One would first identify appropriate islet cell transplant patients. Most commonly, one would identify a Type 1 or Type 2 diabetes patient who is dependent on insulin because of deteriorating or nonfunctioning islet cells. Preferably, one would employ the present invention for newly transplanted patients and one would treat the patients immediately after transplant. Patients who have need of islet cell transplants for other reasons, eg, a traumatic injury or disease targeting the pancreas, would also be suitable candidates.

The studies shown below demonstrate the treatment of murine models for islet transplantation with 2AMD. These models demonstrate that one would be able to treat a mammalian patient, preferably a human patient, in a manner corresponding to the mouse model. NOD mice exhibit a susceptibility to a spontaneous development of insulin-dependent diabetes mellitus. One should note that these mice are genetically identical to each other and transplants of islet cells between mice of the same strain do not require the immunosuppressant drugs that the typical human islet cell transplant requires. Therefore, human and other mammalian patients of the present invention would typically require the use of immunosuppressant drugs, as described below.

Preferably, the patient would be under an insulin treatment regime before islet cell transplantation. After islet cell transplantation, the patient will have a more normalized blood glucose measurement and will typically not require insulin treatment. Islet cell transplantation would be by any of the preferred methods of the prior art.

Following islet cell transplantation, one would typically assess islet graft function and would note diabetes reversal as assessed by blood glucose measurements. One would identify an appropriate glucose measure (normoglycemia) as a non-fasting blood glucose less than 250 mg/dl. Islet destruction is defined as a blood glucose level of greater than 250 mg/dl on at least two consecutive days.

To preserve the positive effects of the islet cells, one would treat the transplant patient with a sufficient amount of 2AMD or 2AMD analog. By “sufficient amount” we mean an amount of a compound, formulation, material, or composition, as described herein effective to achieve a particular biological result. Such results may include, but are not limited to, the treatment of a disease or condition as determined by any means suitable in the art. For instance, an amount is sufficient if it preserves a prolonged period of normoglycemia in an islet cell transplant patient compared to a patent who has not been treated.

Preferably, a human patient would be treated with 50-400 ng of 2AMD or 2AMD analog per patient per day. We have experience with 2MD in human patients and the suggested dose is successfully used for postmenopausal women. We envision that in one embodiment of the present invention, the treatment would go indefinitely as long as one wished to preserve the positive effects of the islet cells.

Preferably, one would use 2AMD or 2AMD analog in any available form, preferably an oral or injected form, as disclosed in U.S. Pat. No. 5,945,410 issued Aug. 31, 1999; U.S. Pat. No. 6,127,559 issued Oct. 3, 2000; and U.S. Pat. No. 6,277,837 dated Aug. 21, 2001. One may wish to add pharmaceutically acceptable fillers and excipients to the therapeutic dose. In another form of the present invention, one may wish to add therapeutically active substances to 2AMD or 2AMD analog. For example, one may wish to add an immunosuppressant to the dose.

One may wish to monitor serum calcium levels after treatment. The examples below disclose that serum calcium levels averaged between 10.7 mg/dl and 10.8 mg/dl in regular chow diets and an average of 10.0 mg/dl with purified diets. We expect that the serum calcium level will be appropriate after human treatment with 2AMD. In terms of protecting a human patient against hypercalcemia, especially expected safe dose range of the present invention is less than 400 ng/day.

One would expect that no adverse effects after 2AMD or 2AMD analog treatment will be observed. Most particularly, one would not expect to see adverse effects on food consumption, weight gain or comparative body weights DeLuca et al. J. Bone Min. Res. 26:538-545, 2011.

The goal of the treatment of the present invention is a prolonged reversal of hyperglycemia. The experimental evidence in the murine model shows at least 130 additional days of continuing normoglycemia following 2AMD treatment compared to the untreated control. We expect that treatment with the compounds of the present invention will extend a human islet cell transplant beyond the current limits. More than 90% of islet cell recipients revert to insulin dependence within five years after islet cell transplantation. The method of the present invention will allow a prolonged life for the transplant. When compared to human longevity, the “at least 130 days”, in the mouse model would be an increased transplant survival of at least several years beyond the non-treated control. “Increased transplant survival” may be measured in several ways. Most typically, one would asses blood glucose. A blood glucose measurement of >250 mg/dl on at least 2 consecutive days would indicate islet destruction. A non-fasting blood glucose less <250 mg/dl indicates a normal and appropriate blood glucose measurement. Normal glucose level is typically 110 ng/100 ml. In other words, if the patient has to revert to insulin to keep a blood glucose measurement in line, the islet cell transplant has failed.

In a preferred form of the present invention, the human patient would also be treated with immunosuppressant as well as 2AMD or 2AMD analog. Preferable immunosuppressive drugs include daclizumab (ZENAPAX), sirolimus (RAPAMUNE), and tacrolimus (PROGRAF).

### EXAMPLES

### Mice

Female NOD and NOD.SCID mice were obtained from The Jackson Laboratory (Bar Harbor, Me.). All mice were maintained at the University of Wisconsin-Madison, Department of Biochemistry animal facility under specific pathogen-free conditions and exposed to 12 h light-dark cycles. The mice were housed in plastic cages lined with cornhusk shavings and consumed distilled water ad libitum. All experimental protocols were approved by the University of Wisconsin Research Animal Resources Center Committee Review Board and conform to national guidelines for animal usage in research.

### Murine Models for Islet Transplantation

Female NOD mice were allowed to develop T1D spontaneously. Upon development of T1D, mice were treated with insulin pellet therapy (LinBit, Canada) and were allocated to either a control group or treatment with 2AMD. Within seven to fourteen days of control or 2AMD treatment, the insulin pellet was removed and either 800 or 400 islet equivalents from female NOD.SCID donors were transplanted under the renal capsule. Following transplantation, islet graft function and diabetes reversal were assessed with blood glucose measurements three times per week. Normoglycemia was defined as non-fasting blood glucose levels less than 250 mg/dl. Islet destruction was defined on the basis of blood glucose levels of >250 mg/dl on at least 2 d consecutively.

### Diabetes Monitoring and Glucose Analysis

NOD mice were screened three times per week for glucosuria using reagent strips (Diastix, Bayer Corporation, Elkhart, Ind.). Mice that tested positive for glucosuria were tested for hyperglycemia using blood from the distal tail. Blood glucose was measured using a One Touch Ultra glucometer (LifeScan, Milpitas, Calif.). Animals with glucose levels greater than 300 mg/dL were diagnosed as diabetic.

### Administration of 2AMD

Mice were treated with either 400 picograms of 2AMD/mouse/day or with vehicle. 2AMD was administered by addition of the analog in the vehicle to LabDiet 5K52 (PMI Nutrition, Brentwood, Miss.). In order to deliver the designated dose of 2AMD, a stock solution of the analog was dissolved in EtOH, vortexed for 3 min, and the analog concentration was determined by spectrophotometry. Based on pre-determined diet consumption, diet was prepared to provide 400 pg of 2AMD/mouse/day. The diets were solidified with molten agar (USB Corporation, Cleveland, Ohio). Controls were fed diet prepared as described above but the vehicle containing no 2AMD. Mice in each group were fed either 2AMD diet or vehicle diet from the day of diabetes determination until the completion of the study.

### Serum Calcium Analysis

Blood was collected from the orbital sinus 3 weeks after initiation of control or 2AMD treatment and every 3 weeks thereafter. Blood samples were spun at 2,938 g for 15 min, followed by a second and third spin at 16,883 g, each for 2 min. Serum calcium levels were determined by atomic absorption in 0.1% LaCl3.

### Isolation of Pancreatic Islets

Under ketamine and Xylazine anesthesia, pancreata were distended through the common bile duct with 3 mL of a customized solution of collagenase and Dispase (Vitacyte LLC, Indianapolis, Ind.) prepared in Hanks Buffered Salt Solution. Each infused pancreas was excised, transferred to a conical tube, and incubated in a water bath at 37° C. for 18 minutes. Islets were separated by a Ficoll density gradient (Mediatech, Inc., Manassas, Va.) and were manually selected under a dissecting microscope. Images of all isolated islets were obtained using Metamorph software and spatially calibrated using pixel conversion into mm derived from a scored transparent grid image. The calibrated images were then transferred to ImageJ software for morphometric analysis. The measurements were exported to an excel spreadsheet customized to quantitate each islet sample from the image analysis using the standard characteristics of total islet number per size class and islet equivalents. Islet equivalent is defined as standardized islet unit corresponding to the volume of a 150 mm diameter sphere.

### Results

### 2AMD Inhibits T1D Recurrence in Transplanted Mice.

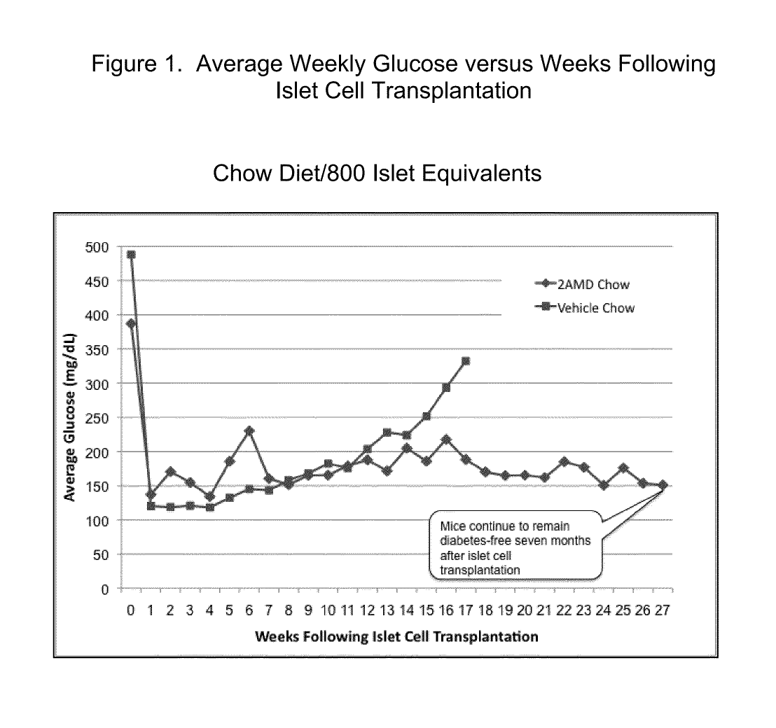

100% of the vehicle-treated mice developed hyperglycemia by 14 weeks following islet cell transplantation. By comparison, treatment with 2AMD completely abrogates the autoimmune response with 100% diabetes reversal and 100% indefinite survival of syngeneic islet grafts (Table 2 and FIG. 1).

TABLE 2
Duration of Normoglycemia Following Islet Cell Transplantation Chow-
based Diet/800 Islet Equivalents

| | Transplanted Islet | Duration of Normoglycemia |
|---|---|---|
| Study Group | Equivalents | (Days) |
| Vehicle Control | 800 | 110 |
| (2mice) | | |
| 400 picograms | 800 | 230 + (continuing without |
| 2AMD/mouse/day | | recurrence of diabetes) |
| (2 mice) | | |

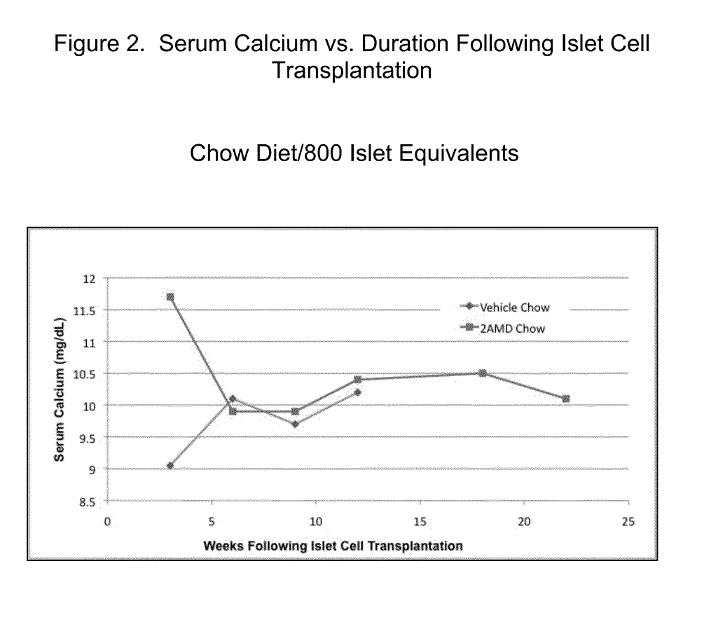

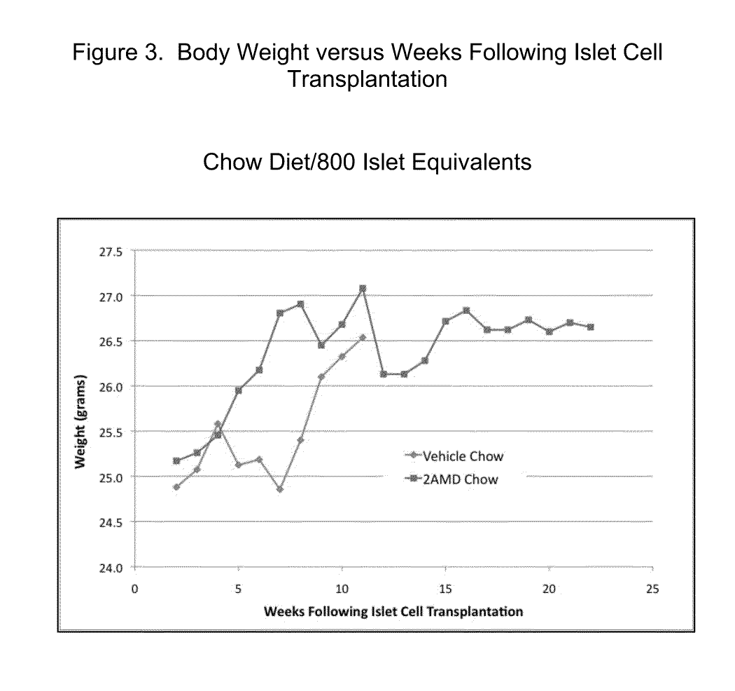

Mice treated with 400 pg/day of 2AMD averaged serum calcium of 10.3 mg/dl compared to 9.8 mg/dl in control mice (FIG. 2). No adverse effects of 2AMD were observed as revealed by food consumption, weight gain and comparative body weights (FIG. 3). Also, 2AMD combined with a purified diet inhibits T1D recurrence in mice transplanted with half the amount of beta cell mass needed to produce long-term normoglycemia in a chow-based diet.

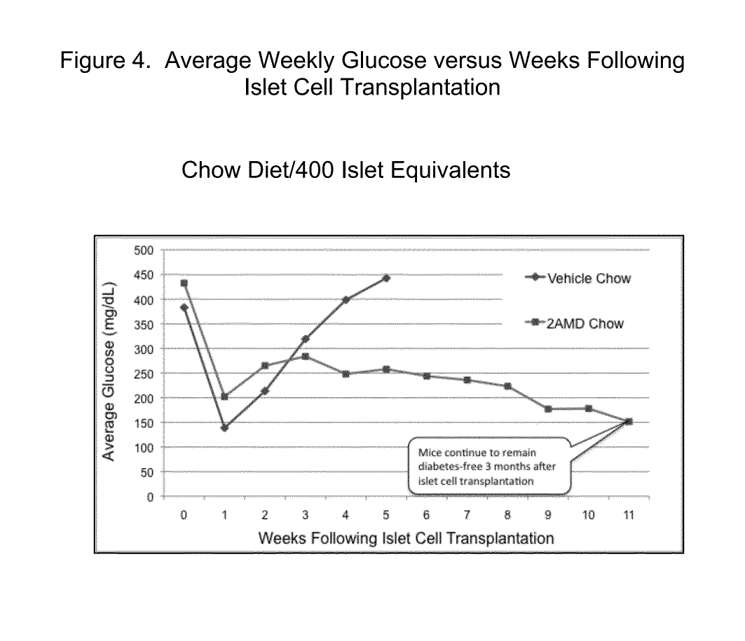

100% of vehicle treated mice fed chow and receiving half the islet mass of 400 islet equivalents developed hyperglycemia by 4 weeks following islet cell transplantation. However, treatment with 2AMD in a chow-based diet prolonged reversal of hyperglycemia for 83 days with continuing normoglycemia at the time of patent submission. Using a purified diet, 100% of the vehicle-treated mice remain normoglycemia 93 days following transplantation of 400 islet equivalents. The combined treatment of 2AMD and a purified diet completely abrogated the autoimmune response with 100% diabetes reversal and 100% indefinite survival of syngeneic islet grafts sustaining more than 230 days of normoglycemia despite the reduced islet mass (Table 3 and FIG. 4).

TABLE 3
Duration of Normoglycemia Chow-based versus Purified Diets/400 Islet
Equivalents

| | | Transplanted | Duration of |
|---|---|---|---|
| Study Group | Diet | Islet Equivalents | Normoglycemia (days) |
| Vehicle Control | Chow | 400 | 28 |
| (3 mice) | | | |
| 400 picograms | Chow | 400 | 83 |
| 2AMD/mouse/day | | | Continuing without |
| (2 mice) | | | diabetes recurrence |
| Vehicle Control | Purified | 400 | 65 |
| (2mice) | | | |
| 400 picograms | Purified | 400 | 230 |
| 2AMD/mouse/day | | | Continuing without |
| (2 mice) | | | diabetes recurrence |

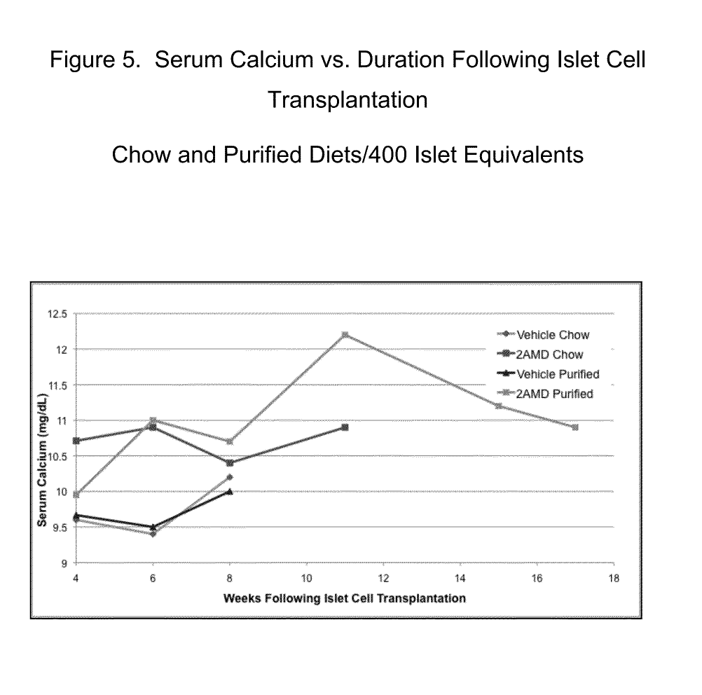

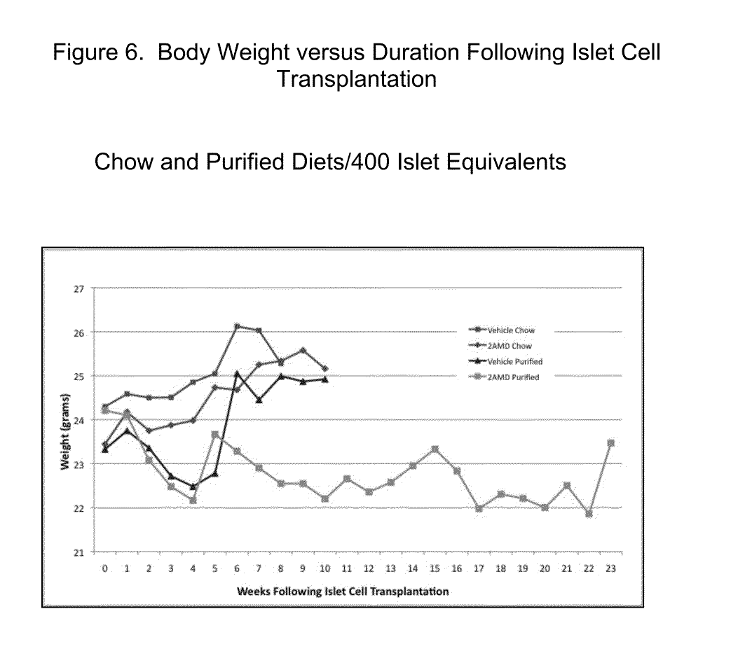

Mice treated with 400 pg/day of 2AMD averaged serum calcium levels of 10.7 mg/dl and 10.8 mg/dl with chow and purified diets respectively compared to an average of 10.0 mg/dl in both control groups (FIG. 5). The reduced food consumption, weight gain, and comparative body weights observed in the 2AMD purified diet group were attributable to increased hydration of two dietary components that produced early satiety. Replacement of these components at week 22 resulted in a significant increase in food consumption and weight gain (FIG. 6).

### CONCLUSIONS

2AMD is a selective 1,25(OH)₂D₃ analog that inhibits recurrent autoimmune responses and enhance long-term graft survival in a model of islet transplantation into diabetic NOD mice. This finding, together with the fact that 2AMD provides immunoprotection, indicates that these analogs are novel candidates for treatment of autoimmune type 1 diabetes and for preventing the recurrence of autoimmune destruction of transplanted islets. In particular, 2AMD completely abrogates the autoimmune response with 100% diabetes reversal and 100% indefinite survival of syngeneic islet grafts. When combined with a purified diet, 2AMD allowed engraftment and long-term survival of half the islet mass required to sustain normoglycemia in a chow-based diet. Therefore 2AMD or 2MD can be used to allow long-term survival and function of islet cells following transplantation.

### REFERENCES

C M Kiekhaefer, et. al., 2α-Methyl-19-nor-(20S)-1,25-dihydroxyvitamin D3 protects the insulin 2 knockout non-obese diabetic mouse from developing type 1 diabetes without hypocalcaemia, Clinical and Experimental Immunology, 2011, 166 (3): 325-32.
DeLuca H F, et al. The vitamin D analogue 2MD increases bone turnover but not BMD in postmenopausal women with osteopenia: Results of a 1-year phase 2 double-blind, placebo-controlled, randomized clinical trial, J. Bone Min. Res. 2011, 26:538-545.

1. A method of treating islet cell transplant patients, comprising the steps of
a) identifying an islet cell transplant patient,
b) treating the patient with a sufficient amount of 2AMD or 2AMD analog such that a prolonged period of normoglycemia occurs compared to a patient who has not been treated, and
c) observing a prolonged period of normoglycemia.

2. The method of claim 1 wherein the 2AMD or 2AMD analog is selected from the group of 2AMD and 2MD.

3. The method of claim 1 comprising the additional step of treating the patient with insulin before the islet cell transplant.

4. The method of claim 1 comprising the additional step of monitoring serum calcium levels after the 2AMD or 2AMD analog treatment.

5. The method of claim 1 wherein the prolonged period is at least 1 year after transplant compared to an untreated control.

6. The method of claim 5 wherein the prolonged period is at least 2 years.

7. The method of claim 5 wherein the prolonged period is at least 3 years.

8. The method of claim 1 comprising the additional step of treating the patient with immunosuppressants.

9. The method of claim 1 wherein the serum calcium level of the patient does not exceed 10.8 mg/dl during the treatment of step (b).

10. A composition for treatment of islet cell transplant patient comprising 50-400 ng/day of 2AMD or 2AMD analog.

11. A composition for treatment of islet cell transplant patient comprising 50-400 ng/day of 2AMD or 2AMD analog and at lease one immunosuppressant.

12. The composition of claim 11, wherein the immunosuppressant is selected from the group consisting of daclizumab (ZENAPAX), sirolimus (RAPAMUNE) and tacrolimus (PROGRAF).
